SEQUENCE LISTING (26 sequences)

SEQ ID NO: 1 - DNA, 20 bp, Artificial Sequence
  hIL15 forward primer, fully synthetic
gcaatgttccatcatgttcc

SEQ ID NO: 2 - DNA, 21 bp, Artificial Sequence
  hIL15 reverse primer, fully synthetic
gcctcctacaatacaatacga

SEQ ID NO: 3 - DNA, 20 bp, Artificial Sequence
  hCXCL1 forward primer, fully synthetic
ctgaacagtgacaaatccaa

SEQ ID NO: 4 - DNA, 22 bp, Artificial Sequence
  hCXCL1 reverse primer, fully synthetic
ggggttgacatttcaaaaagaa

SEQ ID NO: 5 - DNA, 21 bp, Artificial Sequence
  hCCL2 forward primer, fully synthetic
tgagactaacccagaaacatc

SEQ ID NO: 6 - DNA, 21 bp, Artificial Sequence
  hccl2 reverse primer, fully synthetic
cttgaagatcacagcttcttt

SEQ ID NO: 7 - DNA, 20 bp, Artificial Sequence
  IL1beta forward primer, fully synthetic
cattgctcaagtgtctgaag

SEQ ID NO: 8 - DNA, 20 bp, Artificial Sequence
  IL1beta reverse primer, fully synthetic
ttcatctgtttagggccatc

SEQ ID NO: 9 - DNA, 20 bp, Artificial Sequence
  CXCL2 forward primer, fully synthetic
ccaaccatgcataaaagggg

SEQ ID NO: 10 - DNA, 15 bp, Artificial Sequence
  CXCL2 reverse primer, fully synthetic
ggggcgctcctgctg

SEQ ID NO: 11 - DNA, 20 bp, Artificial Sequence
  PTGIS forward primer, fully synthetic
agcttccacattacagcccc

SEQ ID NO: 12 - DNA, 20 bp, Artificial Sequence
  PTGIS reverse primer, fully synthetic
aggagaagtcgaggagaccc

SEQ ID NO: 13 - DNA, 20 bp, Artificial Sequence
  TGFb2 forward primer, fully synthetic
cgaaactgtctgcccagttg

SEQ ID NO: 14 - DNA, 20 bp, Artificial Sequence
  tgfb2 reverse primer, fully synthetic
tgtagaaagtgggcgggatg

SEQ ID NO: 15 - DNA, 20 bp, Artificial Sequence
  CXCL14 forward primer, fully synthetic
ctaagatgaccatgcgccct

SEQ ID NO: 16 - DNA, 20 bp, Artificial Sequence
  CXCL14 reverse primer, fully synthetic
aatgcggcatatactggggg

SEQ ID NO: 17 - DNA, 20 bp, Artificial Sequence
  SERPINE1 forward primer, fully synthetic
gcaaggcacctctgagaact

SEQ ID NO: 18 - DNA, 20 bp, Artificial Sequence
  SERPINE1 reverse primer, fully synthetic
gggtgagaaaaccacgttgc

SEQ ID NO: 19 - DNA, 21 bp, Artificial Sequence
  Progerin forward primer, fully synthetic
gttgaggacgacgaggatgag

SEQ ID NO: 20 - DNA, 22 bp, Artificial Sequence
  Progerin reverse primer, fully synthetic
cagttctgggggctctgggctc

SEQ ID NO: 21 - DNA, 20 bp, Artificial Sequence
  hIL1A forward primer, fully synthetic
tgagtcagcaaagaagtcaa

SEQ ID NO: 22 - DNA, 20 bp, Artificial Sequence
  hIL1A reverse primer, fully synthetic
gattggcttaaactcaaccg

SEQ ID NO: 23 - DNA, 21 bp, Artificial Sequence
  IL6 forward primer, fully synthetic
ctgcaagagacttccatccag

SEQ ID NO: 24 - DNA, 23 bp, Artificial Sequence
  IL6 reverse primer, fully synthetic
agtggtatagacaggtctgttgg

SEQ ID NO: 25 - DNA, 17 bp, Artificial Sequence
  beta-actin forward primer, fully synthetic
agagctagctgcctgac

SEQ ID NO: 26 - DNA, 18 bp, Artificial Sequence
  beta-actin reverse primer, fully synthetic
ggatgccacaggactcca

(19) United States
(12) Patent Application Publication
(10) Pub. No.: US 2022/0031762 A1
(43) Pub. Date: Feb. 3, 2022

(54) USE OF ENDOTHELIAL PROGENITOR CELLS IN REJUVENATING THE MICROVASCULATURE, PREVENTING AGING AND TREATING AGE-RELATED DISEASES

(71) Applicant: SHENZHEN UNIVERSITY, Shenzhen (CN)

(72) Inventors: Baohua LIU, Shenzhen (CN); Shimin SUN, Shenzhen (CN)

(21) Appl. No.: 17/461,094

(22) Filed: Aug. 30, 2021

Related U.S. Application Data
(63) Continuation-in-part of application No. PCT/CN2019/076488, filed on Feb. 28, 2019.

(51) Int. Cl. A61K 35/51; C12N 5/071

(52) U.S. Cl. CPC A61K 35/51; C12N 5/0692

(57) ABSTRACT

Use of endothelial progenitor cells in the manufacture of a medicament for rejuvenating neovascularization capacity, ameliorating aging features, preventing aging, extending lifespan, and/or treating progeria and/or age-related diseases. A method for rejuvenating neovascularization capacity, ameliorating aging features, preventing aging, extending lifespan, and/or treating progeria and/or age-related diseases, includes: administering a pharmaceutically effective amount of EPCs to a subject in need thereof.

### CROSS-REFERENCE TO RELATED APPLICATIONS

This application is a continuation-in-part of International Patent Application No. PCT/CN2019/076488 with a filing date of Feb. 28, 2019, designating the United States, now pending. The content of the aforementioned application is incorporated herein by reference.

### BACKGROUND

### Technical Field

The present invention relates to endothelial progenitor cells (EPCs) and their role in preventing aging, extending lifespan and treating age-related diseases. In particular, the present invention relates to use of endothelial progenitor cells in clinical progeria treatment.

### Description of the Related Art

The statements herein only provide background information related to the present application, and do not necessarily constitute prior art.

Aging represents the largest risk factor for many age-related diseases, as exemplified by vascular dysfunction and cardiovascular diseases (CVDs). The blood vessel consists of the tunica intima (composed of endothelial cells; ECs), the tunica media (composed of vascular smooth muscle cells; VSMCs) and the tunica adventitia (consisting of connective tissue). The endothelium separates the vessel wall from the blood flow and has an irreplaceable role in regulating vascular tone and homeostasis. Age-related functional decline in ECs and VSMCs is a main cause of CVDs. ECs secrete various vasodilators and vasoconstrictors that act on VSMCs and induce blood-vessel contraction and relaxation. For instance, nitric oxide (NO) is synthesized from L-arginine by endothelial NO synthase (eNOS) in ECs and is released on VSMCs to induce blood-vessel relaxation. When ECs become senescent or dysfunctional, vasoconstrictive, pro-coagulative and pro-inflammatory cytokines are released; this effect reduces NO bioavailability and in turn increases vascular intimal permeability and EC migration. Despite advances in the understanding of the mechanisms of endothelial dysfunction, it is unclear whether it directly triggers organismal aging.

Accumulating data suggest that the mechanisms underlying normal aging are similar to those governing Hutchinson-Gilford progeria syndrome (HGPS)—a premature aging syndrome in which affected patients typically succumb to CVDs. HGPS is predominantly caused by an a c. 1824 C>T, p. G608G mutation in LMNA gene, which activates an alternate splicing event and generates a 50-amino-acid-truncated form of lamin A, referred to as progerin. The murine Lmna^{G609G}, which is equivalent to LMNA^{G608G} in humans, causes aging phenotypes resembling HGPS. It has been shown that progerin targets SMCs and causes blood vessel calcification and atherosclerosis. Recent work by two groups showed that SMC-specific progerin knock-in mice are healthy and have a normal lifespan, but suffer from blood-vessel calcification, atherosclerosis and shortened lifespan when crossed to Apoe−/− mice. In contrast to SMCs, the contributing roles of the vascular endothelium (VE) to systemic/organismal aging are elusive.

Endothelial progenitor cells (EPCs) mainly exist in the bone marrow. Upon VE injury, cytokines and growth factors, such as VEGF, SDF-1, G-CSF and estrogen, mobilize EPCs to the peripheral circulation. The EPCs then seed at the injury site and promote repair via neovascularization. An age-related decline in the number and function of EPCs is a main reason for decreased VE repair capacity. Progeria models exhibit depleted stem cells, including mesenchymal stem cells (MSCs), epithelial stem cells, muscular stem cells and hematopoietic stem cells (HSCs). Questions remain as to whether EPCs also decline in progeria and if so, whether this decline causally accelerates aging. To address these issues, we generated a conditional progerin (Lmna^{G609G}) knock-in (KI) model, i.e. Lmna^{f/f} mice. In combination with E2A-Cre and Tie2-Cre mice, we aimed to investigate the roles of the VE dysfunction and the EPCs to systemic aging.

### SUMMARY OF THE INVENTION

Vascular dysfunction is one of the typical characteristics of aging, but its contributing roles to systemic aging is lacking experimental evidence. Accumulating data suggest that mechanisms underlying aging are similar to those governing Hutchinson-Gilford progeria syndrome (HGPS), a premature aging syndrome in which affected patients typically succumb to cardiovascular diseases (CVDs). Here, we generated a knock-in mouse model with the causative HGPS Lmna^{G609G} mutation. Using the Lmna^{f/f} and Tie2-Cre mice, we showed that endothelial-specific dysfunction compromises the microvasculature and neovascularization and accelerates aging in multiple tissues/organs. Most importantly, endothelial-specific dysfunction shortens lifespan in Lmna^{f/f}; TC mice. Mechanistically, single-cell transcriptomic analysis of murine lung endothelial cells (MLECs) revealed a significant up-regulation of genes that regulate inflammation including Il6, Il8, Il15, Cxcl1 and Il1α etc. Further determined by FACS analysis and neovascularization assay, we observed that the number and function of EPCs in the bone marrow decline in Lmna^{f/f}; TC mice compared to Lmna^{f/f} control mice. Replenishing wild-type EPCs rejuvenates neovascularization capacity, ameliorates aging features and extends lifespan in progeria mice. These data reveal that endothelial dysfunction triggers systemic aging and highlight EPC therapy as a potential anti-aging strategy and clinical progeria treatment.

In one aspect, the present invention provides use of endothelial progenitor cells (EPCs) in the manufacture of a medicament for rejuvenating neovascularization capacity, ameliorating aging features, preventing aging, extending lifespan, and/or treating progeria and/or age-related diseases. Preferably, the age-related diseases are cardiovascular diseases and/or osteoporosis. More preferably, the cardiovascular diseases are atherosclerosis and/or heart failure.

In another aspect, the present invention provides a method for rejuvenating neovascularization capacity, ameliorating aging features, preventing aging, extending lifespan, and/or treating progeria and/or age-related diseases, comprising administering a pharmaceutically effective amount of EPCs to a subject in need thereof. Preferably, the age-related diseases are cardiovascular diseases and/or osteoporosis. More preferably, the cardiovascular diseases are atherosclerosis and/or heart failure.

### BRIEF DESCRIPTION OF THE DRAWINGS

FIGS. 1A-D show single-cell transcriptomic profiles of CD31⁺ MLECs. FIG. 1A: Purity analysis of sorted CD31⁺ MLECs by FACS. FIG. 1B shows t-SNE projection of CD31⁺ cells revealed four clusters: endothelial cells (ECs), B lymphocytes (B-like), T lymphocytes (T-like) and Macrophages (Mφ-like). FIG. 1C shows Marker gene expression in the four clusters: ECs (Cd31, Cd34, Cdh5), B-like (Ly6d, Cd22, Cd81), T-like (Cd3d, Cd3e, Cd28) and Mφ-like (Cd14, Cd68, Cd282). FIG. 1D shows Heatmap showing marker gene expression levels in E2A and Flox mice.

FIGS. 2A-E show that single-cell transcriptomic analysis indicates an inflammatory response and cardiac dysfunction in progeroid ECs. FIG. 2A shows t-SNE projection of Lmna^{G609G/G609G} (G609G) and Lmna^{f/f} (Flox) CD31⁺ MLECs, according to transcriptomic data. FIGS. 2B-D show GO and KEGG pathway enrichment of differentially expressed genes between G609G and Flox cells. Lmna^{G609G/G609G} MLECs show enrichment in genes that regulate the inflammatory response (FIG. 2C) and genes related to heart dysfunction (FIG. 2D). FIG. 2E shows quantitative PCR analysis of altered genes observed in (FIG. 2C) and (FIG. 2D) in human umbilical vein endothelial cells (HUVECs) with ectopic expression of progerin or wild type LMNA. Data represent the means±s.e.m. *P<0.05, *P<0.01, *P<0.001 (Student's t test).

FIGS. 3A-F show endothelial-specific dysfunction in progeria mice. FIGS. 3A-B show H&E staining of thoracic aorta sections from (FIG. 3A) Lmna^{f/f}; TC and (FIG. 3B) Lmna^{G609G/G609G} and Lmna^{f/f} control mice showing intima-media thickening. Scale bar, 20 μm. FIG. 3C shows acetylcholine (ACh)-induced thoracic aorta vasodilation in Lmna^{f/f}; TC and Lmna^{f/f} control mice. **P<0.01. FIG. 3D shows ACh-induced thoracic aorta vasodilation in Lmna^{G609G/G609G} and control mice. **P<0.01. FIG. 3E shows Sodium nitroprusside (SNP)-induced thoracic aorta vasodilation in Lmna^{G609G/G609G} and control mice. FIG. 3F shows eNOS level in thoracic aorta sections from Lmna^{f/f}; TC and control mice. Scale bar, 20 μm. All data represent the means±s.e.m. P values were calculated by Student's t test.

FIGS. 4A-D show reduced capillary density and defective neovascularization. FIG. 4A shows immunofluorescence staining (left) and quantification (right) of CD31⁺ gastrocnemius muscle in Lmna^{f/f}; TC and Lmna^{f/f} mice. Scale bar, 50 μm. FIG. 4B shows CD31 immunofluorescent staining in Lmna^{f/f}; TC and Lmna^{f/f} liver. Scale bar, 50 μm. FIG. 4C shows representative microcirculation images (left) and quantification of blood flow recovery (right) following hind limb ischemia in Lmna^{f/f}; TC and Lmna^{f/f} mice. FIG. 4D shows representative transverse sections and quantification of CD31⁺ gastrocnemius muscle 14 days after femoral artery ligation. Scale bar, 50 μm. All data represent the means±s.e.m. P values were calculated by Student's t test.

FIGS. 5A-F show systemic aging phenotypes in Lmna^{f/f}; TC mice. FIG. 5A-C show Masson trichrome staining showing an atheromatous plaque in the aorta (FIG. 5A), smooth muscle cell loss (FIG. 5B) and cardiac fibrosis (FIG. 5C) in Lmna^{f/f}; TC mice. Scale bar, 20 μm. FIG. 5D shows Heart weight and echocardiographic parameters, including heart rate, cardiac output, left ventricular (LV) ejection fraction and LV ejection shortening. FIG. 5E shows Decreased running endurance in Lmna^{f/f}; TC mice. FIG. 5F shows Micro-CT analysis showing a decrease in trabecular bone volume/tissue volume (BV/TV), trabecular number (Tb.N) and trabecular thickness (Tb.Th), and an increase in trabecular separation (Tb.Sp) in Lmna^{f/f}; TC mice. All data represent the means±s.e.m. P values were calculated by Student's t test.

FIGS. 6A-J show that EPCs rejuvenate the microvasculature and extend lifespan in progeria mice. FIG. 6A shows lifespan of EPC-treated and untreated Lmna^{G609G/G609G} mice, Lmna^{f/f}; TC and Lmna^{f/f} mice. FIG. 6B shows body weight of EPC-treated and untreated Lmna^{G609G/G609G} mice, Lmna^{f/f}; TC and Lmna^{f/f} mice. *P<0.05. FIG. 6C shows percent CD133⁺ EPCs in Lmna^{f/f}; TC and Lmna^{f/f} mice. FIG. 6D shows neovascularization assay of CD133⁺ EPCs derived from Lmna^{f/f}; TC and Lmna^{f/f} mice in mice with hind limb ischemia. FIG. 6E shows EPCs from rosa26-rainbow mice rescue hind limb ischemia in Lmna^{f/f}; TC mice. FIG. 6F shows representative immunofluorescence image showing ECs that have differentiated from rosa26-rainbow EPCs. Scale bar, 15 μm. FIGS. 6G-J show representative immunofluorescence images of the liver (FIG. 6G), aorta (FIG. 6H), muscle (FIG. 6I) and lung (FIG. 6J) of Lmna^{f/f}; TC mice after EPC therapy, showing ECs that have differentiated from rosa26-rainbow EPCs. Scale bar, 15 μm. All data represent the means s.e.m. P values were calculated by Student's t test.

FIGS. 7A-D shows generation of Lmna^{f/f} mice and phenotypic analysis of Lmna^{G609G/G609G} mice. FIG. 7A shows schematic illustration of knock-in strategy of Lmna^{f/f} mice harboring Lmna^{G609G} mutation (Lmna 1827C>T). FIG. 7B shows representative photo of Lmna^{G609G/G609G} mice and Lmna^{f/f} control mice. FIG. 7C shows representative immunoblot showing Lamin A, Progerin and Lamin A expression in Lmna^{G609G/+} Lmna^{G609G/G609G} and Lmna^{+/+} control mice. FIG. 7D shows lifespan determination of Lmna^{G609G/+} Lmna^{G609G/G609G} and Lmna^{+/+} mice.

FIGS. 8A-B show single cell transcriptomic analysis of CD31⁺ MLECs. FIG. 8A shows p21^{Cip/Waf1} mRNA levels in Lmna^{G609G/G609G} (G609G) and Lmna^{f/f}(Flox) CD31⁺ MLECs. p21^{Cip/Waf1} was elevated specifically in ECs and MD-like cells isolated from G609G mice. FIG. 8B shows Cd45 and Tie2 levels in G609G and Flox CD31⁺ MLECs. Cd45 expression was lacking in ECs and Tie2 expression was EC-specific.

FIGS. 9A-B show VE-specific progerin expression. FIGS. 9A-B show Progerin and CD31 expression was detected by immunofluorescence staining in aorta (FIG. 9A) and muscle FIG. 9B) tissue of Lmna^{f/f}; TC and Lmna^{f/f} mice.

FIGS. 10A-B show vasodilation analysis of Lmna^{G609G/+} mice. Acetylcholine (ACh)-induced (FIG. 10A) and sodium nitroprusside (SNP)-induced (FIG. 10B) vasodilation in Lmna^{G609G/+} and Lmna^{+/+} control mice.

FIGS. 11A-B show the expression of atherosclerosis-associated (FIG. 11A) and osteoporosis-associated (FIG. 11B) genes in MLEC transcriptomes.

FIG. 12 shows CD133⁺ Endothelial progenitor cells labeled with Dil-acLDL and UEA. The nuclei were counterstained with DAPI. Scale bar, 50 μm.

FIGS. 13A-E show comparison of expression levels of genes that are associated with atherosclerosis (FIG. 13A), arthritis (FIG. 13B), heart failure (FIG. 13C), osteoporosis and amyotrophy (FIG. 13D) in different clusters of cells (FIG. 13E) recovered from the single-cell RNA sequencing.

### DETAILED DESCRIPTION OF THE EMBODIMENTS

The present invention provides use of endothelial progenitor cells (EPCs) in the manufacture of a medicament for rejuvenating neovascularization capacity, ameliorating aging features, preventing aging, extending lifespan, and/or treating progeria and/or age-related diseases, more preferably atherosclerosis and/or heart failure.

In a specific embodiment, the EPCs are CD133⁺ EPCs.

In a specific embodiment, the age-related diseases are characterized by vascular endothelium (VE) dysfunction.

In particular, the VE dysfunction includes a loss of endothelial cells, reduced capillary density and defective neovascularization capacity.

More particularly, the VE dysfunction is caused by progerin.

The present invention also provides a method for rejuvenating neovascularization capacity, ameliorating aging features, preventing aging, extending lifespan, and/or treating progeria and/or age-related diseases, comprising administering a pharmaceutically effective amount of EPCs to a subject in need thereof; preferably, the age-related diseases are cardiovascular diseases and/or osteoporosis, more preferably atherosclerosis and/or heart failure.

In a specific embodiment, the EPCs are CD133⁺ EPCs.

In a specific embodiment, the age-related diseases are characterized by vascular endothelium (VE) dysfunction.

In particular, the VE dysfunction includes a loss of endothelial cells, reduced capillary density and defective neovascularization capacity.

More particularly, the VE dysfunction is caused by progerin.

The present invention will be further demonstrated by the following experimental procedures and examples, which are used only for illustration purpose, but not limiting the scope of the present invention.

Experimental Procedures

Animals

Lmna^{f/f} alleles (Lmna^{G609G} flanked by 2 loxP sites) were generated accordingly. The 5′ and 3′ homology arms were amplified from BAC clones RP23-21K15 and RP23-174J9, respectively. The G609G (GGC to GGT) mutation was introduced into exon 11 in the 3′ homology arm. C57BL/6 embryonic stem cells were used for gene targeting. To obtain ubiquitous progerin expression (Lmna^{G609G/G609G}) Lmna^{f/f} mice were bred with E2A-Cre mice. To obtain VE-specific progerin expression, Lmna^{f/f} mice were bred with 71e2-cre mice. Mice were purchased from Cyagen Biosciences Inc., China, housed and handled in accordance with protocols approved by the Committee on the Use of Live Animals in Teaching and Research of Shenzhen University, China.

Hind Limb Ischemia

Four months old male mice were anesthetized with 4% chloral hydrate (0.20 ml/20 g) by intraperitoneal injection. Hind limb ischemia was performed by unilateral femoral artery ligation and excision, as previously described. In brief, the neurovascular pedicle was visualized under a light microscope following a 1-cm incision in the skin of the left hind limb. Ligations were made in the left femoral artery proximal to the superficial epigastric artery branch and anterior to the saphenous artery. Then, the femoral artery and the attached branches between ligations were excised. The skin was closed using a 4-0 suture line and erythromycin ointment was applied to prevent wound infection after surgery. Recovery of the blood flow was evaluated before and after surgery using a dynamic microcirculation imaging system (Teksgray, Shenzhen, China). Relative blood flow recovery is expressed as the ischemia to non-ischemia ratio. At least three mice were included in each experimental group.

Cell Culture

HEK293 cells and human umbilical vein endothelial cells (HUVECs) were purchased from ATCC. HEK293 cells were cultured in Gibco® DMEM (Life Technologies, USA) supplemented with 10% fetal bovine serum (FBS) at 37° C., 5% CO₂. HUVECs were cultured in Gibco® M199 (Life Technologies, USA) supplemented with 15% FBS, 50 μg/ml endothelial cell growth supplement (ECGS) and 100 μg/ml heparin at 37° C., 5% CO₂. All cell lines used were authenticated by short tandem repeat (STR) profile analysis and were mycoplasma free.

RNA Isolation and Quantitative PCR (Q-PCR) Analysis

Total RNA was extracted from cells or mouse tissues using Trizol® reagent RNAiso Plus (Takara, Japan) and transcribed into cDNA using 5× Primescript RT Master Mix (Takara, Japan), following the manufacturer's instructions. The mRNA levels were determined by quantitative PCR with SYBR Premix Ex Taq 11 (Takara, Japan) detected on a CFX Connected Real-Time PCR Detection System (Bio-Rad). All primer sequences are listed in Table 1.

TABLE 1

| Targets | | |
|---|---|---|
| (Homo) | Forward | Reverse |
| hIL15 | SEQ ID NO. 1: | SEQ ID NO. 2: |
| | GCAATGTTCCATCATGTTCC | GCCTCCTACAATACAA |
| | | TACGA |
| hCXCL1 | SEQ ID NO. 3: | SEQ ID NO. 4: |
| | CTGAACAGTGACAAATCCAA | GGGGTTGACATTTCAA |
| | | AAAGAA |
| hCCL2 | SEQ ID NO. 5: | SEQ ID NO. 6: |
| | TGAGACTAACCCAGAAACAT | CTTGAAGATCACAGCT |
| | C | TCTTT |
| IL1β | SEQ ID NO. 7: | SEQ ID NO. 8: |
| | CATTGCTCAAGTGTCTGAAG | TTCATCTGTTTAGGGC |
| | | CATC |
| CXCL2 | SEQ ID NO. 9: | SEQ ID NO. 10: |
| | CCAACCATGCATAAAAGGGG | GGGGCGCTCCTGCTG |
| PTGIS | SEQ ID NO. 11: | SEQ ID NO. 12: |
| | AGCTTCCACATTACAGCCCC | AGGAGAAGTCGAGGAG |
| | | ACCC |
| TGFb2 | SEQ ID NO. 13: | SEQ ID NO. 14: |
| | CGAAACTGTCTGCCCAGTTG | TGTAGAAAGTGGGCGG |
| | | GATG |
| CXCL14 | SEQ ID NO. 15: | SEQ ID NO. 16: |
| | CTAAGATGACCATGCGCCCT | AATGCGGCATATACTGG |
| | | GGG |
| SERPINE | SEQ ID NO. 17: | SEQ ID NO. 18: |
| 1 | GCAAGGCACCTCTGAGAAC | GGGTGAGAAAACCACGT |
| | T | TGC |
| Progerin | SEQ ID NO. 19: | SEQ ID NO. 20: |
| | GTTGAGGACGACGAGGATG | CAGTTCTGGGGGCTCTG |
| | AG | GGCTC |
| hILlA | SEQ ID NO. 21: | SEQ ID NO. 22: |
| | TGAGTCAGCAAAGAAGTCA | GATTGGCTTAAACTCAA |
| | A | CCG |
| IL6 | SEQ ID NO. 23: | SEQ ID NO. 24: |
| | CTGCAAGAGACTTCCATCCA | AGTGGTATAGACAGGTC |
| | G | TGTTGG |
| β-actin | SEQ ID NO. 25: | SEQ ID NO. 26: |
| | AGAGCTAGCTGCCTGAC | GGATGCCACAGGACTCC |
| | | A |

Protein Extraction and Western Blotting

For protein extraction, cells were suspended in SDS lysis buffer and boiled. Then, the lysate was centrifuged at 12,000×g for 2 min and the supernatant was collected. For Western blotting, protein samples were separated on SDS-polyacrylamide gels, transferred to PVDF membranes (Millipore, USA), blocked with 5% non-fat milk and incubated with the relevant antibodies. Images were acquired on a Bio-Rad system. All antibodies are listed in Table 2.

TABLE 2

| Antibodies | M/R/Rat | | Vendor | Dilution |
|---|---|---|---|---|
| VEGFR2 | rabbit | ab2349 | abcam | 1:100 |
| CD31 | rabbit | ab28364 | abcam | 1:100 |
| SMA | rabbit | ab5694 | abcam | 1:500 |
| CD31 | rat | ab7388 | abcam | 1:50 |
| progerin | mouse | ab66587 | abcam | 1:50 |
| LaminA/C | mouse | ab8984 | abcam | 1:100 |
| Enos | mouse | ab76198 | abcam | 1:100 |
| LaminA | mouse | sc-71488 | Santa Cruz | 1:100 |
| progerin | mouse | ab-81611 | Santa Cruz | 1:50 |
| LaminA/C | rabbit | sc-20681 | Santa Cruz | 1:100 |
| PE-CD31 | rat | 553373 | BD pharmingen | 1:100 |
| PE-CD34 | rat | 551387 | BD pharmingen | 1:250 |
| PE-Flk-1 | rat | 555308 | BD pharmingen | 1:250 |
| PE-CD117 | rat | 553355 | BD pharmingen | 1:250 |
| PE-IgG2b k Isotype | rat | 555848 | BD pharmingen | 1:100 |
| control | | | | |
| Alexa Fluor 488- | rat | 557676 | BD pharmingen | 1:250 |
| IgG2a k Isotype | | | | |
| control | | | | |
| Purified Anti-mouse | rat | 553141 | BD pharmingen | 1:250 |
| CD16/CD32 | | | | |
| APC-CD133 | mouse | 141208 | Biolegend | 1:250 |
| APC-IgG2a k | rat | 400511 | Biolegend | 1:250 |
| Isotype control | | | | |
| Alexa Fluor 488- | mouse | 121908 | Biolegend | 1:250 |
| Flk-1 | | | | |
| Alexa Fluor 488- | rat | 400525 | Biolegend | 1:250 |
| IgG2a k Isotype | | | | |
| control | | | | |
| prominin-1-biotin | mouse | 130-111-353 | Miltenyi Biotec | 1:100 |
| (CD133) | | | | |
| CD34-biotin | mouse | 130-105-830 | Miltenyi Biotec | 1:100 |
| Flk-1-microBeads- | mouse | 130-097-346 | Miltenyi Biotec | |
| Kit | | | | |
| MACS separation | | 130-091-221 | Miltenyi Biotec | |
| buffer | | | | |
| Anti-biotin- | | 130-090-485 | Miltenyi Biotec | 1:4 |
| microBeads | | | | |
| Dil-ac-LDL | | L-3484 | invitrogen | 1:100 |
| FITC-UEA-1 | | L9006-1MG | SIGMA | 1:100 |

Immunofluorescence Staining

Aorta, skeletal muscle and liver tissues were collected from Lmna^{G609G/G609G} Lmna^{+/+}, Lmna^{f/f}; TC and Lmna^{f/f} mice. Frozen sections were prepared and fixed in 4% PFA, permeabilized with 0.3% Triton X-100, blocked with 5% BSA and 1% goat serum, and then incubated with primary antibodies at room temperature for 2 h or at 4° C. overnight. After three washes with PBST, the sections were incubated with secondary antibodies for 1 h at room temperature and then stained with DAPI anti-fade mounting medium. Images were captured under a Zeiss LSM880 confocal microscope. All antibodies are listed in Table 2.

Masson Trichrome Staining

Paraffin-embedded sections of PFA-fixed tissues were dewaxed and hydrated. Staining was then performed using a Masson trichrome staining kit (Beyotime, China). In brief, the sections were dipped in Bouin buffer for 2 h at 37° C., and then successively stained with Celestite blue staining solution, Hematoxylin staining solution, Ponceau's staining solution and Aniline blue solution for 3 minutes. After dehydrating with ethyl alcohol three times, the sections were mounted with Neutral Balsam Mounting Medium (BBI Life Science, China). Images were captured under a Zeiss LSM880 confocal microscope.

Fluorescence Activated Cell Sorting (FACS)

Mice were sacrificed by decapitation. The lungs were then collected, cut into small pieces and then digested with collagenase I (200 U/ml) and neutral protease (0.565 mg/ml) for 1 h at 37° C. The isolated cells were incubated with PE-conjugated anti CD31 antibody for 1 h at 4° C. and then 7-AAD (1:100) for 5 min. CD31-positive and 7-AAD-negative cells were sorted on a flow cytometer (BD biosciences, USA).

Myography

Four months old male mice were anesthetized with 4% chloral hydrate by intraperitoneal injection. Thoracic aortas were collected, rinsed in ice-cold Krebs solution and cut into 2 mm-length rings. Each aorta ring was bathed in 5 ml oxygenated (95% O₂ and 5% CO₂) Krebs solution at 37° C. for 30 min in a myograph chamber (620M, Danish Myo Technology). Each ring was stretched in a stepwise fashion to the optimal resting tension (thoracic aortas to ˜9 mN) and equilibrated for 30 min. Then, 100 mM K+ Krebs solution was added to the chambers to elicit a reference contraction and then washed out with Krebs solution at 37° C. until achieved a baseline. Vasodilatation induced by acetylcholine (Ach) or sodium nitroprusside (SNP) (1 nM to 100 μM) was recorded in 5-HT (2 μM) contracted rings. Data are represented as a percentage of force reduction and the peak of K+-induced contraction. At least three mice were included in each experimental group.

Mice/Human Cytokine Antibody Array

A cytokine assay for mice or human samples (RayBio®) was performed according to the manufacturer's instructions. Briefly, membranes were incubated in blocking buffer for 30 min at room temperature. The samples prepared from serum or cell lysates were added to each membrane and incubated for 4 h at room temperature. After three washes with buffer 1 and two washes with buffer 2, the membranes were reacted with a biotinylated antibody cocktail at 4° C. overnight. After incubation with 1000×HRP-Streptavidin for 2 h, the membranes were again washed three times with buffer 1 and two times with buffer 2 and then visualized using a Bio-Rad detection system. At least three mice were included in each experimental group.

Echocardiography

7-8 months old male mice were anesthetized by isoflurane gas inhalation and then subjected to transthoracic echocardiography (IU22, Philips). Parameters, including heart rate, cardiac output, left ventricular posterior wall dimension (LVPWD), left ventricular end-diastolic dimension (LVEDD), left ventricular end-systolic diameter (LVESD), LV ejection fraction and LV fractional shortening were acquired. At least three mice were included in each experimental group.

Bone Density Analysis

7-8 months old male mice were sacrificed by decapitation. The thigh bone was fixed in 4% PFA at 4° C. overnight. The relevant data were collected by micro-CT (Scanco Medical, pCT100). At least three mice were included in each experimental group.

Endurance Running Test

A Rota-Rod Treadmill (YLS-4C, Jinan Yiyan Scientific Research Company, China) was used to monitor fatigue resistance. Briefly, mice were placed on the rotating lane and the speed of the rotations gradually increased to 40 r/min. When the mice were exhausted, they were safely dropped from the rotating lane and the latency to fall was recorded. At least three mice were included in each experimental group.

10× Genomics Single-Cell-RNA-Sequencing

CD31⁺ cells isolated from murine lung by FACS (>90% viability) were used for single-cell RNA sequencing. A sequence library was built according to the Chromium Single Cell Instrument library protocol. Briefly, single-cell RNAs were barcoded and reverse-transcribed using Chromium™ Single Cell 3′ Reagent Kits v2, then fragmented and amplified to generate cDNAs. The cDNAs were quantified using an Agilent Bioanalyzer 2100 DNA Chip, and the library was sequenced using an Illumina Hiseq PE150 with ˜10-30M raw data assigned for each cell. The reads were mapped to the mouse mm9 genome and analyzed using STAR: >90% reads mapped confidently to genomic regions and >50% mapped to exonic regions. Cell Ranger 2.1.0 was employed to align reads, generate feature-barcode matrices and perform clustering and gene expression analysis. >80,000 mean reads and 900 median genes per cell were obtained. The UMI (unique molecular identifier) counts were used to quantify the gene expression levels and the t-SNE algorithm was used for dimensionality reduction. The cell population was then clustered by k-means clustering (k=4). The Log2FoldChange was the ratio of gene expression of one cluster to that of all other cells. The p-value was calculated using the negative binomial test and the false discovery rate was determined by Benjamini-Hochberg procedure. GO and KEGG enrichment analysis were performed in DAVID version 6.8.

Isolation of CD133⁺ Progenitor Cells

3 months old male mice were sacrificed by decapitation. The femora and tibiae were separated and placed in a 0.5 ml micro-centrifuge tube which had a hole drilled in the bottom. A 1.5 ml micro-centrifuge tube was used to nest the 0.5 ml tube and the pair of tubes was centrifuged at 10,000×g for 15 sec. The bone marrow was suspended in 1 ml red blood cell lysis buffer at room temperature for 5 min, and the suspension was strained successively through a 75-μm and then 40-μm cell strainer (FALCON®, USA). After centrifugation at 300×g at 4° C. for 5 min, the cells were suspended in 500 μl MACS buffer and incubated with 5 μl anti-CD133 antibody (Miltenyi Biotec, Germany) for 10 min. After incubating with 20 μl beads (Miltenyi Biotec, Germany) in 80 μl MACS buffer, CD133⁺ progenitor cells were obtained by magnetic selection. At least three mice were included in each experimental group.

Statistical Analysis

A two-tailed Student's t-test was used to determine statistical significance. All data are presented as the means±s.d. or means±s.e.m. as indicated, and a p value <0.05 was considered statistically significant.

### Example 1 Single-Cell Transcriptomic Analysis Reveals Four Predominant Cell Clusters in CD31⁺ Murine Lung Endothelial Cells (MLECs)

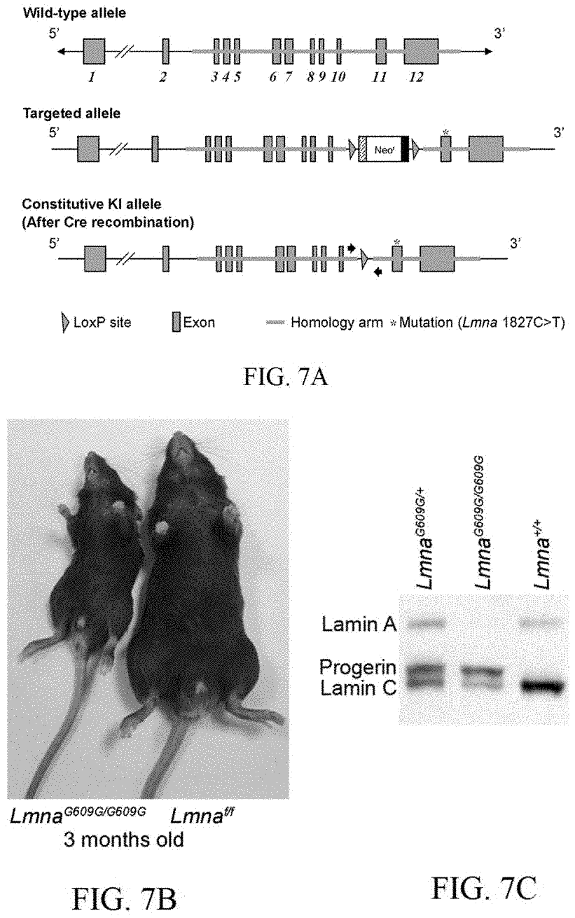

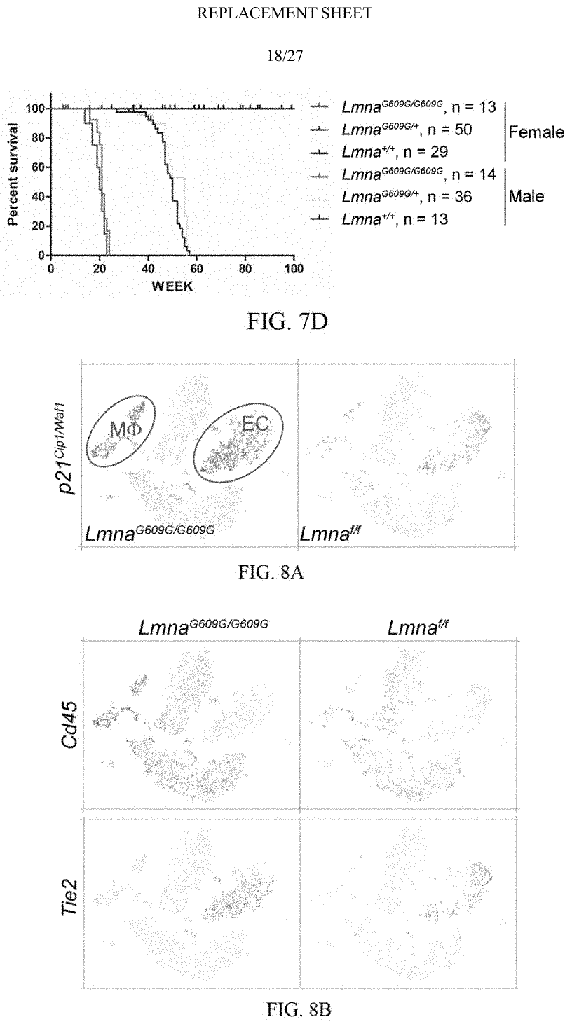

An outstanding question in the field of aging is whether endothelial dysfunction causally triggers systemic aging. The heterogeneity of vascular cells and their close communication with the blood stream, however, renders it difficult to understand the primary function of the VE. The murine Lmna^{G609G} mutation, which is equivalent to human LMNA^{G608G}, causes aging phenotypes in various tissues resembling HGPS. To examine the contributing roles of the VE to systemic aging, we generated a mouse model of conditional progerin knock-in, in which the Lmna^{G609G} mutation was flanked with loxP sites, i.e. Lmna^{f/f} mice (FIG. 7A). The Lmna^{f/f} mice were crossed to E2A-Cre mice, in which Cre recombinase is ubiquitously expressed including germ cells, to generate Lmna^{G609G/G609G} mice. Progerin was ubiquitously expressed in these Lmna^{G609G/G609G} mice, which recapitulated many progeroid features found in HGPS, including growth retardation and shortened lifespan etc. (FIGS. 7B-D).

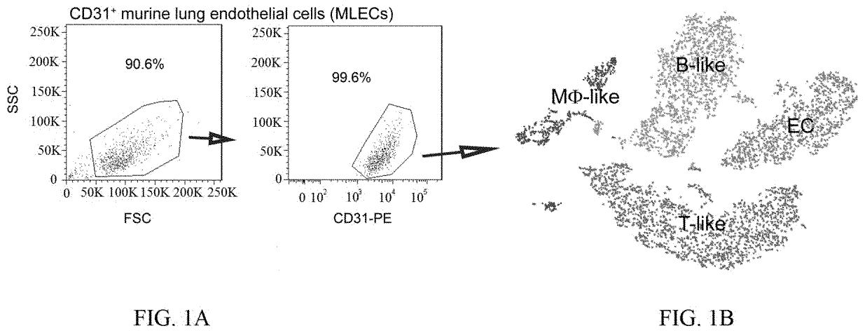

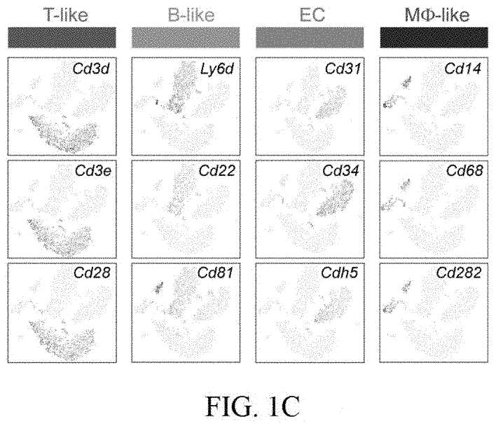

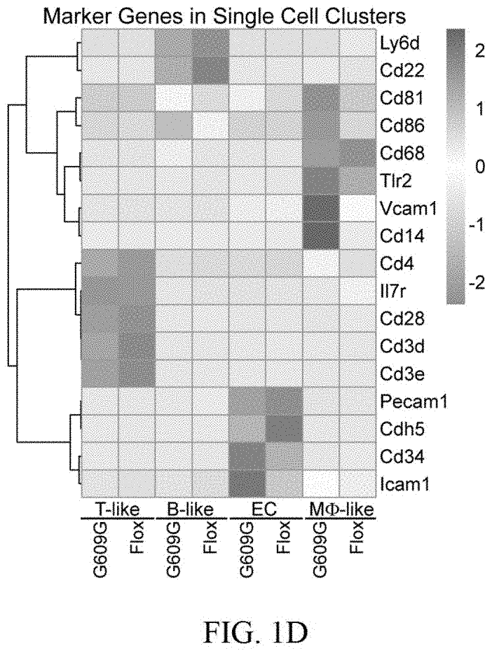

To understand the primary alterations in the VE, we isolated CD31⁺ MLECs from three pairs of Lmna^{G609G/G609G} (G609G) and Lmna^{f/f} (Flox) control mice by FACS (FIG. 1A) and performed 10× Genomics single-cell RNA sequencing. We recovered 6,004 cells (4,137 from G609G and 1,867 from Flox mice) and used the k means clustering algorithm to cluster the cells into four groups (FIG. 1B). As expected, one group exhibited high Cd31, Cd34 and Cdh5 expression, and thus largely represented MLECs. The other three groups, co-purified with CD31V MLECs by FACS, showed relatively lower Cd31 expression (>10-fold lower than MLECs) but high Cd45 expression (FIGS. 8A-B). Further analysis revealed that these clusters most likely contained B lymphocytes (B-like), with high Cd22, Cd81 and Ly6d expression; T lymphocytes (T-like) with high Cd3d, Cd3e and Cd28 expression; and Macrophages (Mφ-like) with high Cd14, Cd68 and Cd282 expression (FIG. 1C). Most of the marker-gene expression levels were comparable between G609G and Flox mice, except for Cd34 and Icam1, which were significantly elevated in G609G ECs, and Cd14 and Vcam1, which were increased in G609G Mφ-like cells (FIG. 1D). Of note, Icam1 and Vcam1 are among the most conserved markers of endothelial senescence and atherosclerosis. Thus, we established a Lmna^{f/f} conditional progerin KI mouse model and revealed a unique EC population for mechanistic study.

### Example 2 Progeroid ECs Exhibit a Systemic Inflammatory Response

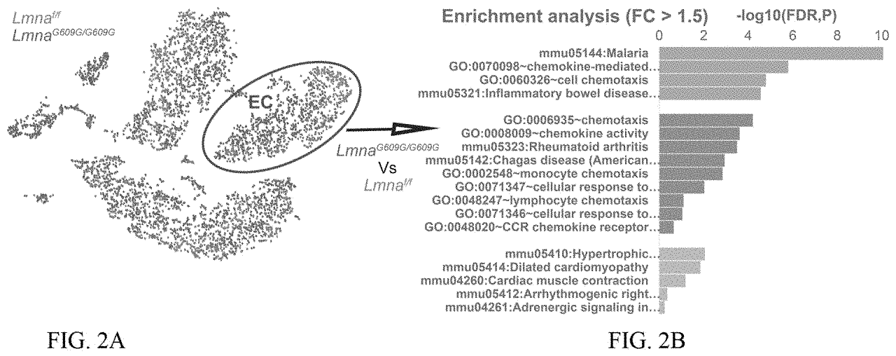

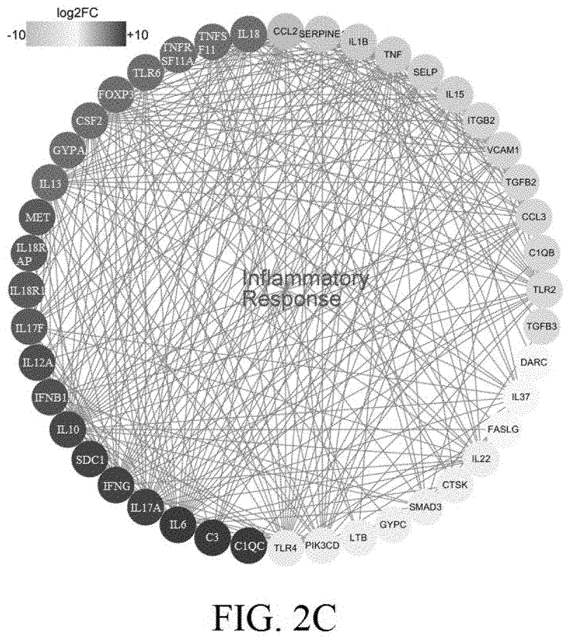

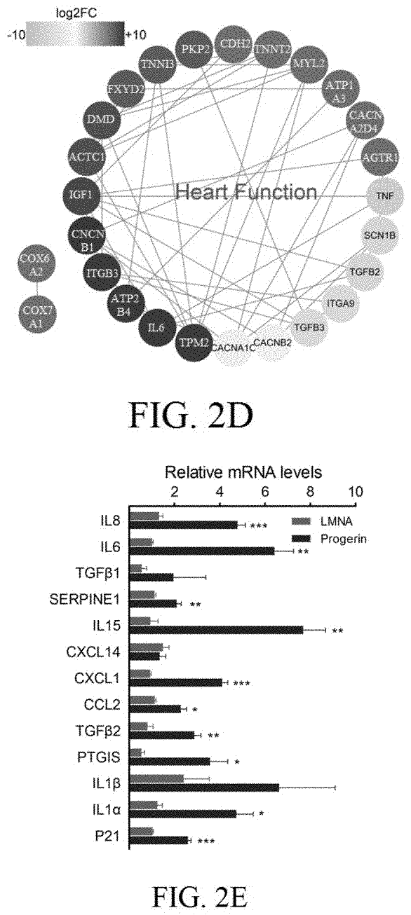

Of the four clusters of CD31⁺ MLECs, ECs and Mφ-like cells showed high levels of p21^{Cip1/Waf1} (FIG. 8A), a typical senescence marker. This finding suggests that these cells are the main target of progerin in the context of aging. Interestingly, a previous study reported that Mφ-specific progerin, achieved by crossing Lmna^{f/+} to Lyz-Cre mice, caused minimal aging phenotypes, implicating that Mφ might have only a minor role in organismal aging. We thus focused on ECs for further analysis. We recovered 899 and 445 ECs from E2A and Flox mice, respectively (FIG. 2A). Genes with >1.5-fold change in expression between these mice were chosen for GO and KEGG analysis. We observed a significant enrichment in the pathways that regulate chemotaxis, immune responses in Malaria and Chagas diseases, inflammatory bowel disease and rheumatoid arthritis and pathways essential for cardiac function (parts FIGS. 2B-D). To confirm this observation, and to exclude paracrine effects from other cell types, we over-expressed progerin in human umbilical vein endothelial cells (HUVECs) and analyzed the representative genes by quantitative PCR. The majority of the examined genes, including IL6, IL8, IL15, CXCL1 and IL1α were significantly up-regulated upon ectopic progerin expression (FIG. 2E). Together, these data suggest that progerin might cause an inflammatory response in ECs, which leads to systemic aging in various organs.

### Example 3 VE Dysfunction Promotes Vasodilation Defects in Progeria Mice

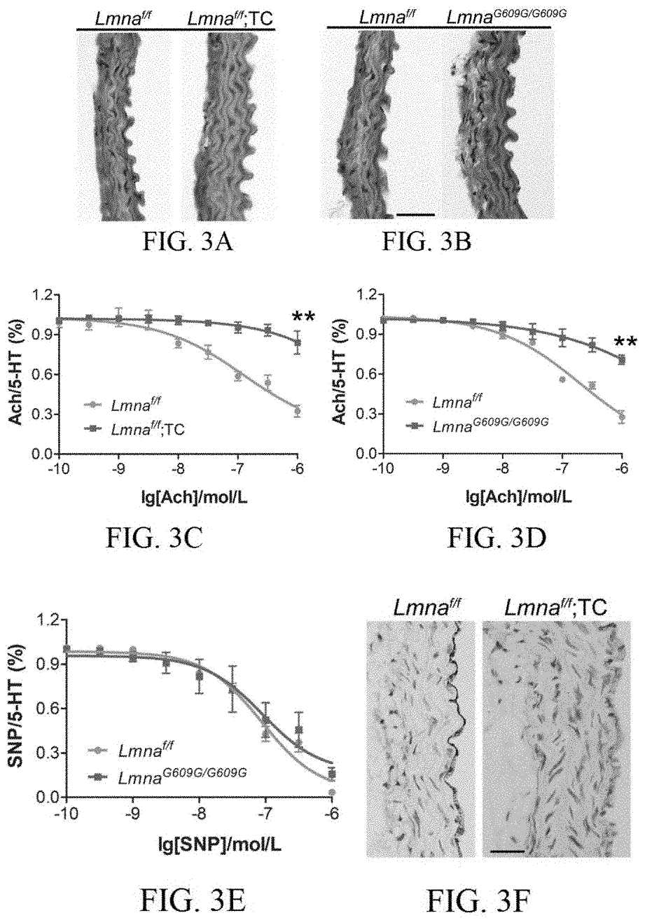

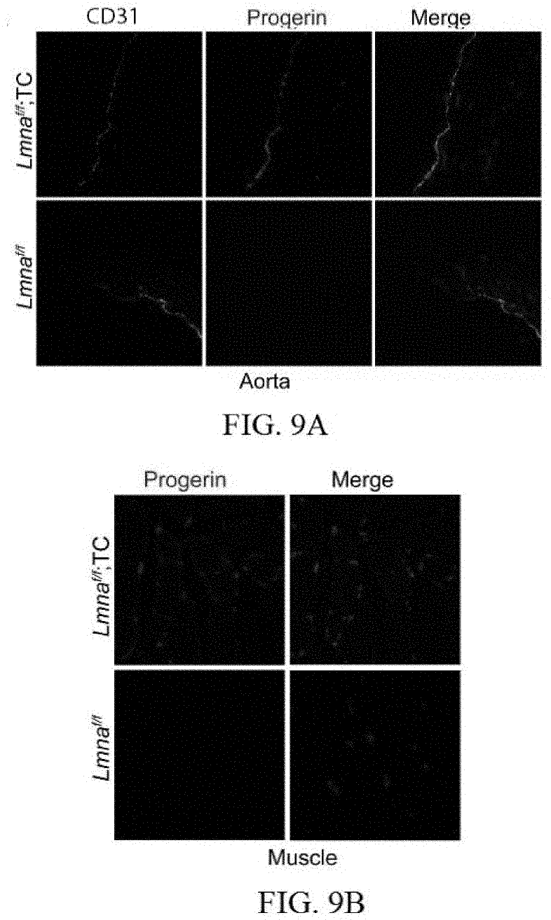

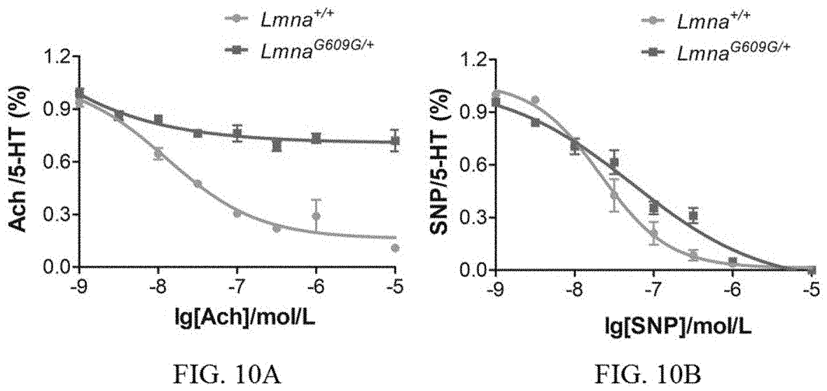

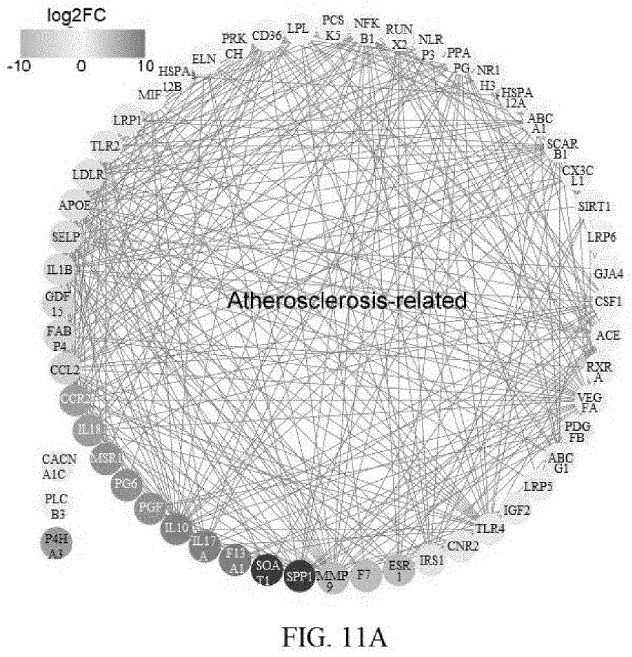

Our single-cell transcriptomic analysis in MLECs and quantitative PCR in HUVECs suggest that the VE have essential roles in systemic aging. To confirm these findings, we crossed the Lmna^{f/f} mice to a Tie2-Cre line, in which Cre recombinase expression is driven by the promoter/enhancer of endothelial-specific Tie2 gene, to generate Lmna^{f/f}; TC mice. Single-cell transcriptome analysis confirmed that Tie2 gene was mainly detected in ECs (FIG. 8B). Consistently, progerin was only observed in the VE of Lmna^{f/f}; TC but not Lmna^{f/f} control mice or other tissues (FIGS. 9A-B). VE-specific progerin induced intima-media thickening in Lmna^{f/f}; TC mice, in a similar manner as Lmna^{G609G/G609G} mice (FIGS. 3A-B). We next performed a functional analysis of the VE based on acetylcholine (Ach)-regulated vasodilation. Ach-induced thoracic aorta relaxation was significantly compromised in Lmna^{f/f}; TC mice (FIG. 3C). Similar defects were observed in Lmna^{G609G/G609G} and Lmna^{G609G/+} mice (FIG. 3D and FIGS. 10A-B), where progerin was expressed in both ECs and SMCs. To gain more evidence supporting VE-specific dysfunction, we examined thoracic aorta relaxation induced by sodium nitroprusside (SNP), which is a SMC-dependent vasodilator. Little difference was observed in thoracic aorta vasodilation in Lmna^{G609G/G609G} and Lmna^{G609G/+} compared to Lmna^{f/f} control mice (FIG. 3E and FIGS. 10A-B), supporting that the VE dysfunction is a key contributor of vasodilation defects in progeria mice. As NO is the most potent vasodilator, we examined eNOS levels in thoracic aorta of Lmna^{f/f}; TC and Lmna^{f/f} control mice. As expected, the level of eNOS was significantly reduced in Lmna^{f/f}; TC mice compared to Lmna^{f/f} control mice (FIG. 3F). Thus, the data confer a VE-specific dysfunction in progeria mice.

### Example 4 Progeria Mice Show Defective Neovascularization Following Ischemia

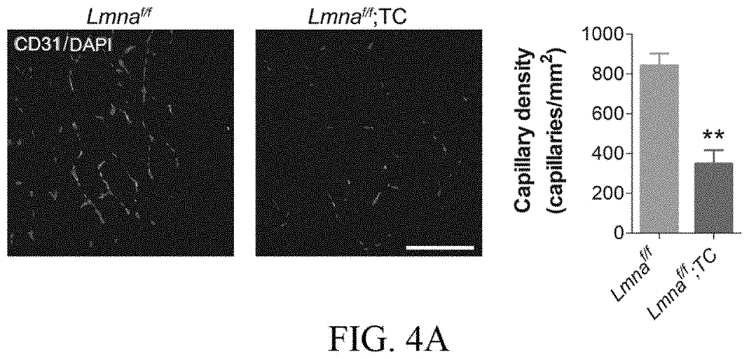

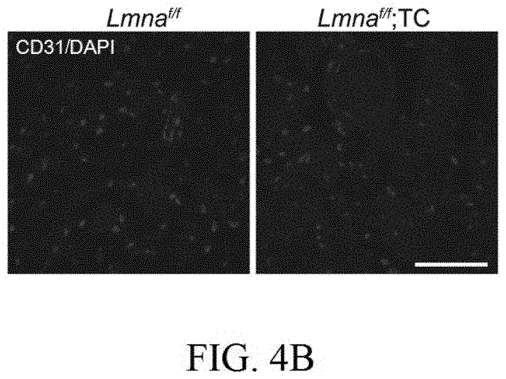

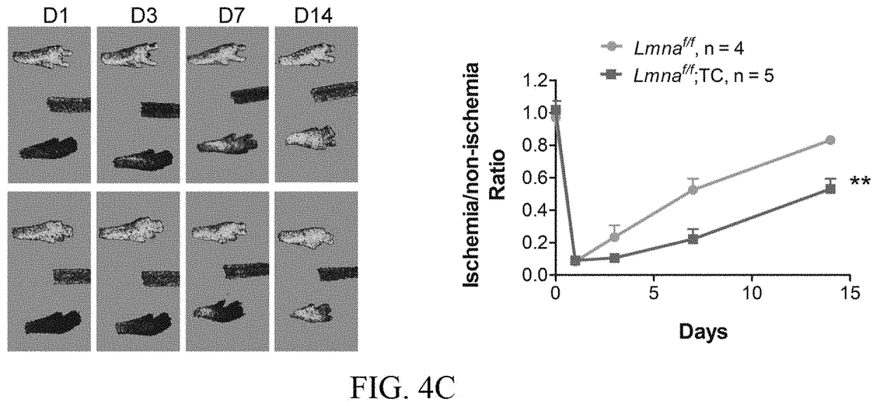

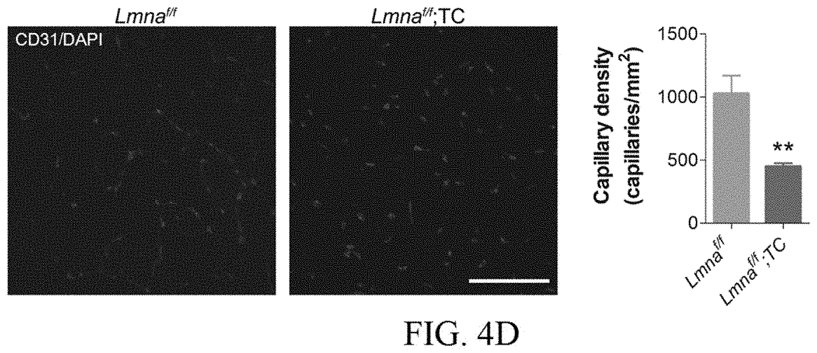

Reduced capillary density and neovascularization capacity are both characteristics of vascular aging. We thus examined the microvasculature in various tissues of Lmna^{f/f}; TC mice by immunofluorescence staining. We observed a significant loss in CD31⁺ ECs in Lmna^{f/f}; TC mice compared to controls (FIGS. 4A-B). We further examined ischemia-induced neovascularization ability in Lmna^{f/f}; TC mice following femoral artery ligation. Indeed, limb perfusion after ischemia was significantly blunted in Lmna^{f/f}; TC mice compared to controls (FIG. 4C). Histological analysis confirmed that the defect in blood-flow recovery in Lmna^{f/f}; TC mice was a reflection of an impaired ability to form new blood vessels in the ischemic region (FIG. 4D). Taken together, Lmna^{f/f}; TC mice are characterized by a loss of ECs, a reduced capillary density and defective neovascularization capacity.

### Example 5 Endothelial Dysfunction is a Causal Factor of Systemic Aging

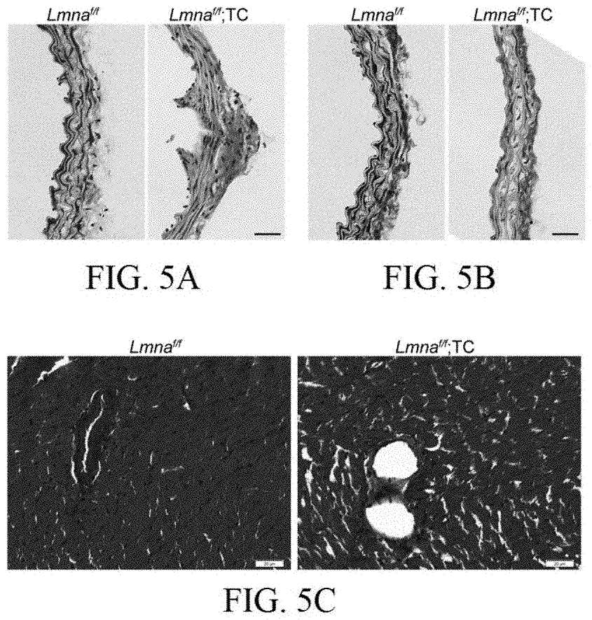

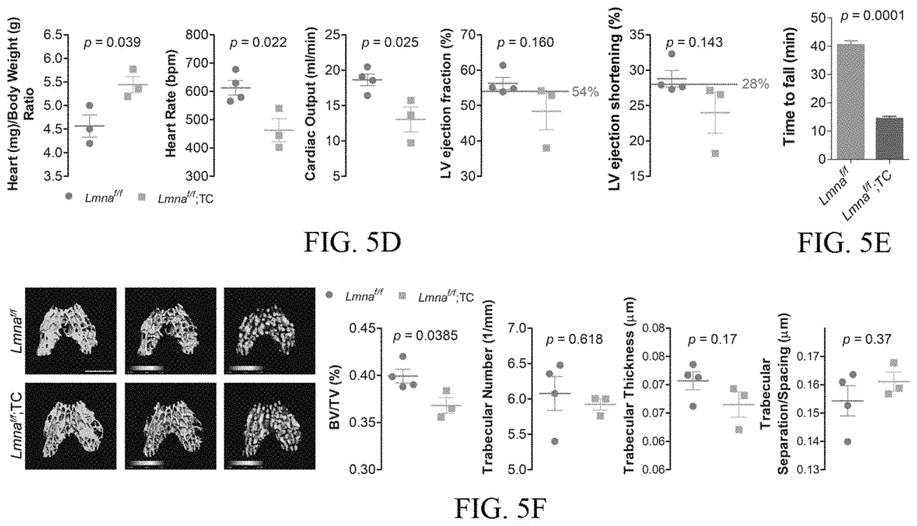

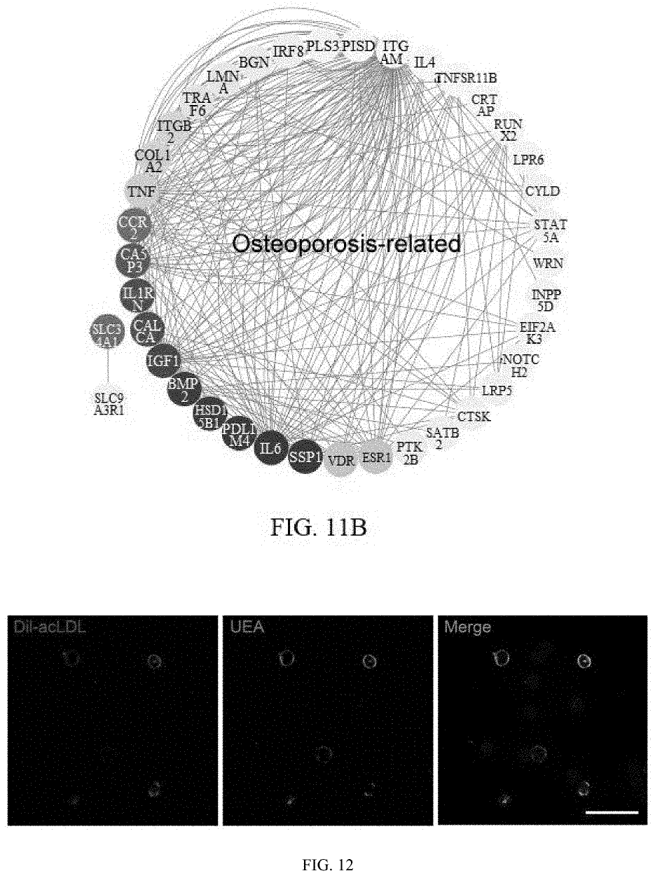

Our single-cell transcriptomic data implicated heart dysfunction in Lmna^{G609G/G609G} mice (FIGS. 2A-E). We also observed a significant correlation with gene alterations associated with atherosclerosis and osteoporosis in Lmna^{G609G/G609G} ECs (the Online Mendelian Inheritance in Man database) (FIGS. 11A-B). We thus reasoned that endothelial dysfunction might trigger systemic aging. Strikingly, atherosclerosis was prominent in Lmna^{f/f}; TC mice (FIG. 5A; aorta atheromatous plaque observed in all eight examined mice), as well as severe fibrosis in the arteries and hearts (FIG. 5B-C); both are typical features of aging. Moreover, the heart/body weight ratio was significantly increased in Lmna^{f/f}; TC compared to Lmna^{f/f} control mice (FIG. 5D). Echocardiography confirmed that the heart rate and cardiac output were significantly reduced in 7-8-month old Lmna^{f/f}; TC compared to Lmna^{f/f} control mice. Both the left ventricular ejection fraction (LVEF) and fractional shortening (LVFS) were below the normal values of healthy mice, which are 54% and 28% respectively. We also found that the running endurance was largely compromised in Lmna^{f/f}; TC mice (FIG. 5E), which is likely a reflection of amyotrophy and/or heart dysfunction. Finally, micro-computed tomography (micro-CT) identified a decrease in trabecular bone volume/tissue volume (BV/TV), trabecular number (Tb.N) and trabecular thickness (Tb.Th), but an increase of trabecular separation (Tb.Sp) in Lmna^{f/f}; TC mice (FIG. 5F), indicative of osteoporosis, which is also a hallmark of aging. Together, these results implicate that endothelial dysfunction, at least in the context of progeria, acts as a causal factor of systemic aging.

### Example 6 EPCs Rejuvenate the Microvasculature, Ameliorate Aging and Extend Lifespan

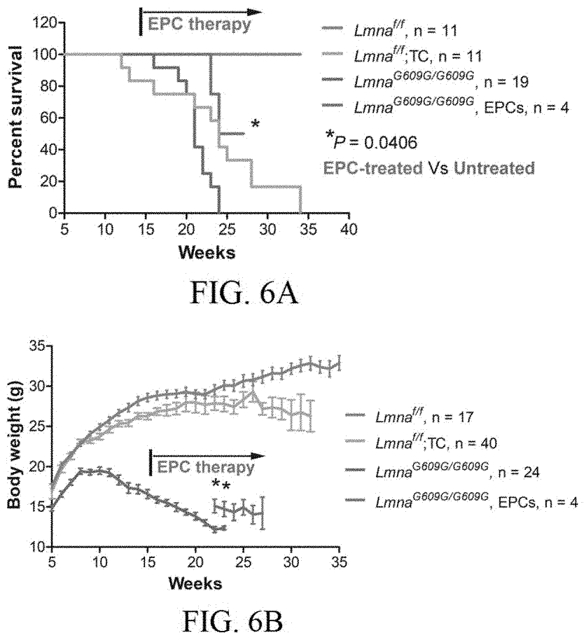

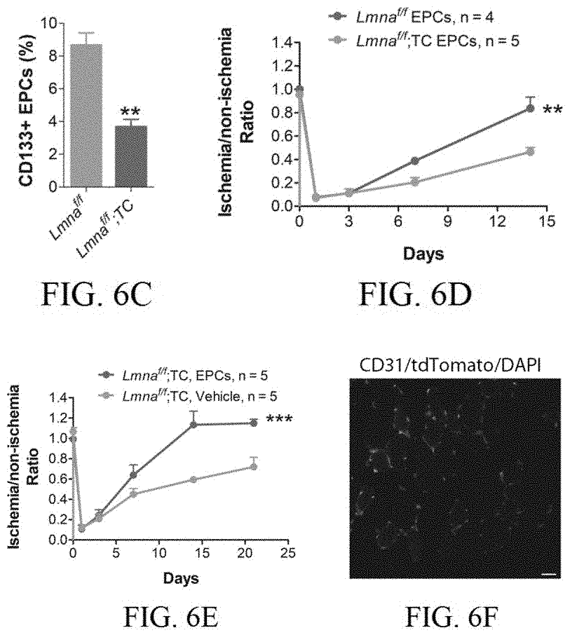

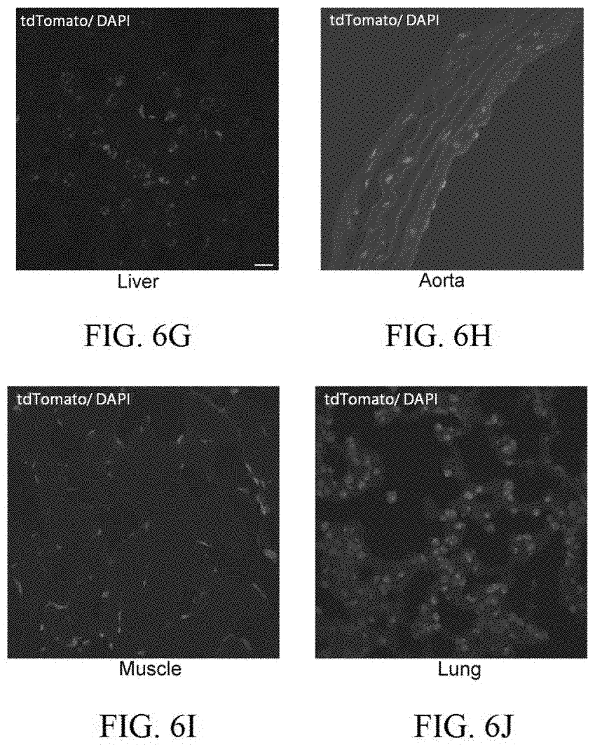

The VE-specific dysfunction not only accelerated aging in various tissues/organs, but also shortened the median lifespan in Lmna^{f/f}; TC mice (24 weeks), to a similar extent as Lmna^{G609G/G609G} mice (21 weeks) (FIG. 6A). While Lmna^{G609G/G609G} mice suffered from body-weight loss from 8 weeks of age, Lmna^{f/f}; TC mice only showed a slight drop in body weight (FIG. 6B). These data suggest that body-weight loss itself is a less likely primary contributing factor to progeria than endothelial dysfunction.

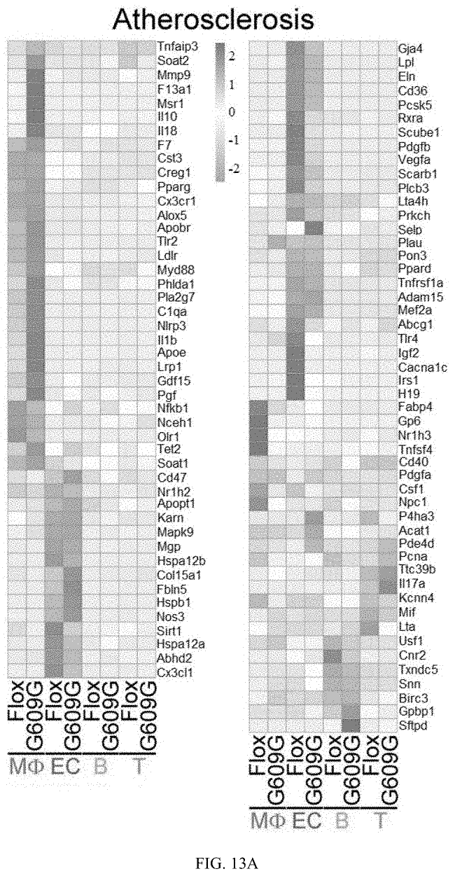

CD133⁺ mononuclear cells are enriched in the bone marrow and are potential EPCs that are essential for vascular hemostasis. We purified CD133⁺ EPCs from Lmna^{f/f}; TC mice and Lmna^{f/f} control mice by FACS and studied the functional relevance in VE dysfunction and aging. Here, we found that >30% of the freshly isolated EPCs were positive for low density lipoproteins (as indicated by Dil-acLDL labeling) and Ulex europaeus agglutinin 1 (UEA-1) (FIG. 12), indicating the endothelial potential of the EPCs. We then analyzed EPCs in progeria mice. The number of EPCs dropped by up to 50% in Lmna^{f/f}; TC mice compared to Lmna^{f/f} control (FIG. 6C). The neovascularization ability of Lmna^{f/f}; TC EPCs was compromised more than 30% (FIG. 6D). We next asked whether EPC decline contributes to the neovascularization defect in progeria mice. On-site injection of CD133⁺ EPCs isolated from rosa26-rainbow mice (tdTomato labeled) completely restored the neovascularization defect in Lmna^{f/f}; TC mice, and histological analyses confirmed the presence of donor-derived ECs in the regenerated vasculature of Lmna^{f/f}; TC mice (FIGS. 6E-F).

We then asked whether EPCs have a causal role in accelerating aging and shortening lifespan in progeria mice. To this end, we injected (via the tail vein) 1×106 MACS (Magnetic-activated cell sorting)-purified EPCs from rosa26-rainbow mice into Lmna^{G609G/G609G} mice. EPCs were administered from 15 weeks before the earliest death event in Lmna^{G609G/G609G} mice and repeated every week. Two of the EPC-treated mice were still alive at 27 weeks-of-age and were sacrificed for histological analysis. Donor-derived ECs were detected by fluorescence microscopy in the liver, muscle, aorta and lung (FIGS. 6G-J, tdTomato labeled). Capillary density (CD31⁺ gastrocnemius muscle) significantly increased from 347.2±121.5 (untreated) to 581.5±85.6 (EPC-treated). More importantly, age-related body-weight loss was significantly attenuated upon EPC therapy in Lmna^{G609G/G609G} mice (FIG. 6B), and the median lifespan was extended from 21 to 27 weeks (FIG. 6A). A reduced systemic inflammatory response was confirmed by an antibody array detecting protein factors in the blood serum (FIG. 12). Altogether, these data suggest that progerin-caused endothelial dysfunction and systemic aging are partially, if not entirely, attributable to EPC decline.

### DISCUSSION

Mounting evidence supports that endothelial dysfunction is a conspicuous marker for vascular aging and CVDs. Whether endothelial dysfunction primarily triggers organismal aging, however, is elusive. The murine Lmna^{G609G} mutation, equivalent to the LMNA^{G608G} found in humans with HGPS, causes premature aging phenotypes in various tissues/organs, thus providing an ideal model for studying aging mechanisms at both the tissue and organismal level. Data from the Lmna^{G609G} model have suggested that SMCs are the primary cause of vascular diseases, such as atherosclerosis. Interestingly, a recent study showed that the specific expression of Lmna^{G609G} in SMCs only causes atherosclerosis and shortens lifespan in atherosclerosis-prone apolipoprotein E-deficient (Apoe−/−) mice. The researchers also found that macrophage-specific Lmna^{G609G} knock-in mediated by Lyz-Cre merely affects aging and lifespan. Here, we used Tie2-Cre mice to generate a VE-specific Lmna^{G609G} model. These mice exhibited vascular dysfunction, accelerated aging and a shortened lifespan to a similar extent to the whole body Lmna^{G609G} model. In support of our findings, Foisner et al. recently reported that the VE-cadherin promoter-driven endothelial-specific expression of progerin in a transgenic line causes cardiovascular abnormalities and shortened lifespan. The data from both our study and that of Foisner strongly suggest that, as the largest secretory organ, the VE is pivotal in regulating systemic aging and lifespan.

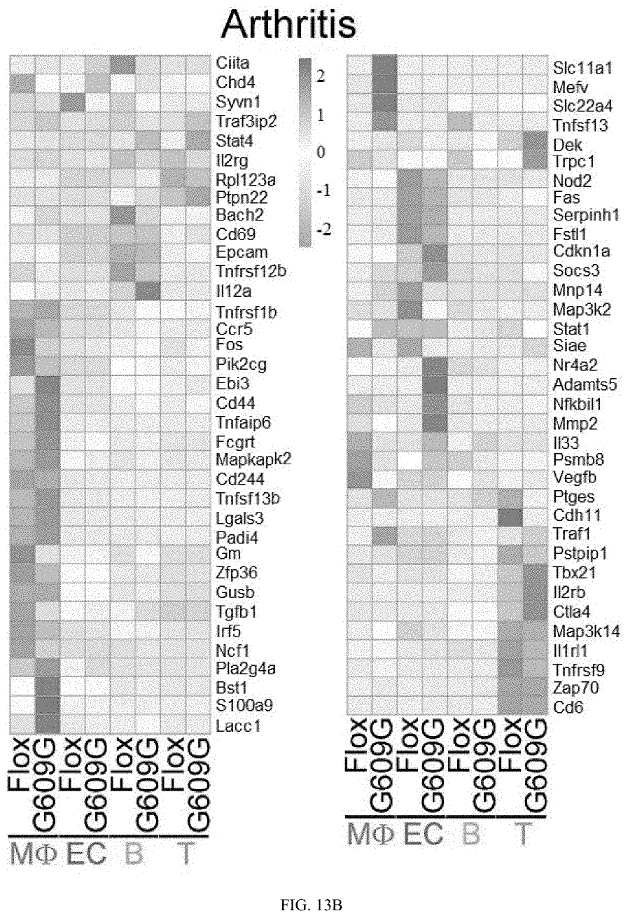

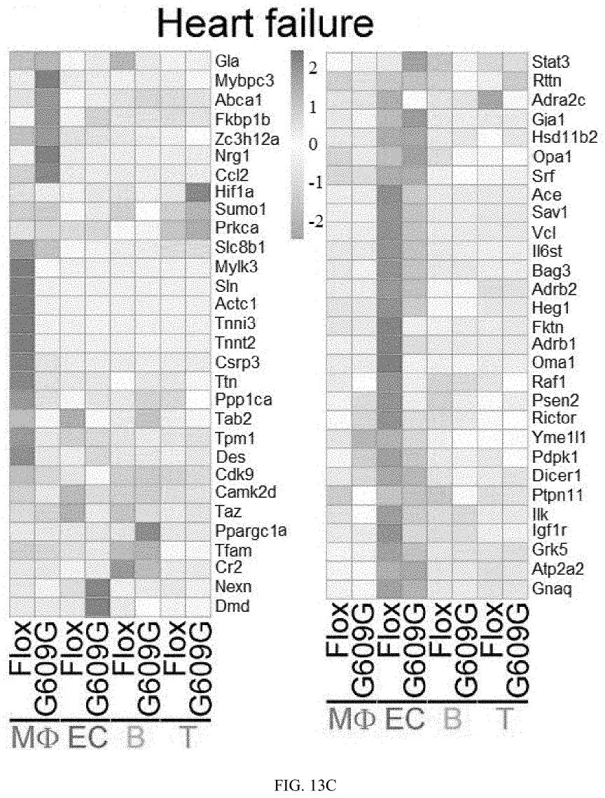

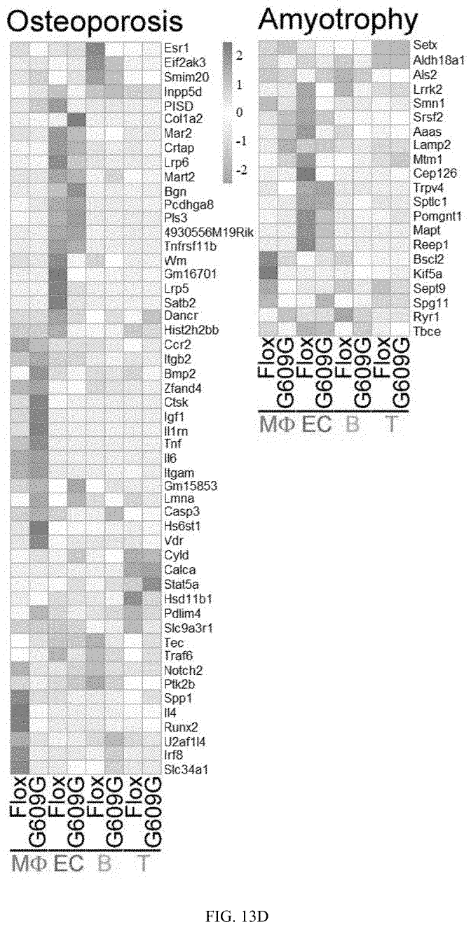

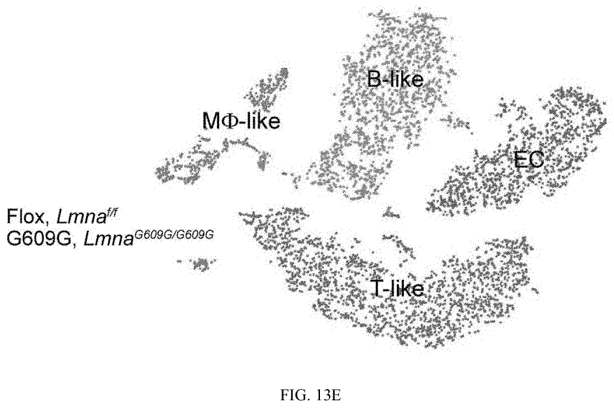

One limitation in the understanding of mechanisms of VE dysfunction is the vascular cell heterogeneity and the lack of appropriate in vitro system for ECs. Here, we took advantage of single-cell RNA sequencing technique to analyze the transcriptomes of MLECs. Surprisingly, although >95% purity was achieved by FACS, MLECs isolated by CD31-immunofluorescence labeling turned out to be a mixture of cells, including ECs, T-like, B-like and Mφ-like cells. It is unclear whether these cells are T cells, B cells and Mφ cells that express low level of CD31, or are transdifferentiated from ECs. Nevertheless, this finding suggests that one can't just purify CD31⁺ cells and pool them together for further mechanistic study, otherwise might get misleading conclusion. Indeed, we compared the expression of genes that are associated with atherosclerosis, arthritis, heart failure, osteoporosis or amyotrophy (the Online Mendelian Inheritance in Man database) between progeroid and control in all four clusters. An obvious alteration of these genes/pathway was observed mainly in ECs and Mφ-like cells (FIGS. 13A-E). As we did single-cell transcriptomic analysis in Lmna^{G609G/G609G} mice, it is difficult to separate cell-autonomous and paracrine effects among different cell populations. In future study, it is worth to do a similar analysis in Lmna^{f/f}; TC MLECs. The data will be useful to study the paracrine effect of ECs on the other cell populations.

The stem-cell theory of aging dictates that the number and functional decline of stem cells directly leads to defective tissue regeneration and consequently organismal aging. EPCs, MSCs and HSCs represent 3 stem-cell populations found in the bone marrow, of which the latter two have implicated clinical potential. We previously showed that the number and function of MSCs and HSCs decline in another progeria mouse model, Zmpste24−/− mice; however, we didn't observe any beneficial effect when MSCs from healthy donors was transplanted into Zmpste24−/− mice by tail vein injection. Consistent with the rapid decline of HSCs and MSCs, we found that the number and function of EPCs, represented by CD133⁺ mononuclear cells, significantly declined in progeria mice compared to healthy controls. Remarkably, EPC transplantation via tail vein injection improved the microvasculature, attenuated body-weight loss, and extended lifespan in progeria mice. To our knowledge, this study provides the first evidence to support the potential of stem-cell therapy in progeria treatment. We thus consider it worthwhile to optimize the conditions of this therapy to maximize the rescue effect elicited by EPCs, and to screen for chemicals that increase the number, improve the function and promote the mobilization of EPCs. Indeed, various drugs clinically used to treat CVDs, such as statins and PPARγ agonists, can mobilize EPCs from the bone marrow to peripheral circulation and enhance endothelial repair. Thus, further investigation is warranted to examine whether these drugs can slow down aging and promote longevity.

Collectively, we reveal that VE dysfunction is a trigger of systemic aging and is also a risk factor for age-related diseases like atherosclerosis, heart failure and osteoporosis. It suggests that many clinically used drugs and molecules that target VE might serve as good candidates in the treatment of age-related diseases other than CVDs. Likewise, the findings in EPCs implicate great potentials of stem-cell-based therapeutic strategy for progeria as well as in anti-aging applications.

What is claimed is:

1. Use of endothelial progenitor cells in the manufacture of a medicament for rejuvenating neovascularization capacity, ameliorating aging features, preventing aging, extending lifespan, and/or treating progeria and/or age-related diseases.

2. Use according to claim 1, wherein the endothelial progenitor cells are CD133⁺ endothelial progenitor cells.

3. Use according to claim 1, wherein the age-related diseases are cardiovascular diseases and/or osteoporosis.

4. Use according to claim 3, wherein the cardiovascular diseases are atherosclerosis and/or heart failure.

5. Use according to claim 1, wherein the progeria and/or age-related diseases are characterized by vascular endothelium dysfunction.

6. Use according to claim 5, wherein the vascular endothelium dysfunction comprises: a loss of endothelial cells, reduced capillary density and defective neovascularization capacity.

7. Use according to claim 5, wherein the vascular endothelium dysfunction is caused by progerin.

8. A method for rejuvenating neovascularization capacity, ameliorating aging features, preventing aging, extending lifespan, and/or treating progeria and/or age-related diseases, comprising administering a pharmaceutically effective amount of EPCs to a subject in need thereof.

9. The method according to claim 8, wherein the age-related diseases are cardiovascular diseases and/or osteoporosis.

10. The method according to claim 8, wherein the age-related diseases are atherosclerosis and/or heart failure.

11. The method according to claim 8, wherein the EPCs are CD133⁺ EPCs.

12. The method according to claim 8, wherein the age-related diseases are characterized by vascular endothelium dysfunction; particularly, the vascular endothelium dysfunction includes a loss of endothelial cells, reduced capillary density and defective neovascularization capacity.

13. The method according to claim 12, wherein the vascular endothelium dysfunction is caused by progerin.
